(19) United States
(12) Patent Application Publication
(10) Pub. No.: US 2010/0056628 A1
(43) Pub. Date: Mar. 4, 2010

(54) Preservative compositions

(76) Inventors: Richard F. Stockel, Bridgewater, NJ (US); Anthony Joseph Sawyer, Oakton, VA (US)

Correspondence Address: ANTHONY J. SAWYER, 3264 PONY RIDGE WAY, OAKTON, VA, 22124, US

(21) Appl. No.: 12/589,155

(22) Filed: Oct. 19, 2009

Related U.S. Application Data
(63) Continuation-in-part of application No. 11/517,147, filed on Sep. 7, 2006.
Application No. 11/517,147 is a continuation-in-part of application No. 11/633,231, filed on Dec. 4, 2006.
Application No. 11/633,231 is a continuation-in-part of application No. 12/585,695, filed on Sep. 22, 2009.
Application No. 12/585,695 is a continuation-in-part of application No. 12/583,919, filed on Aug. 27, 2009.
(60) Provisional application No. 61/196,455, filed on Oct. 17, 2008.

(51) Int. Cl. A01N 37/12; C07C 229/02; A01P 15/00

(52) U.S. Cl. 514/551; 560/155; 560/169

(57) ABSTRACT

Green and naturally derived biocides such as N^{α}—(C1-C22) alkanoyl di basic amino acid alkyl (C1 to C22) ester salt cationic molecules can be combined with an anionic molecule not generally considered as having significant biocidal activity to provide antimicrobial, antibacterial, and/or antifungal properties with multifunctional benefits including preservative or self-preserving activity.

### REFERENCE TO RELATED APPLICATIONS

This application claims the benefit of provisional application Ser. No. 61/196,455, filed Oct. 17, 2008. Included for reference in its entirety is continuation in part Ser. No. 11/517,147, filed Sep. 7, 2006, and continuation in part Ser. No. 11/633,231 filed on Dec. 4, 2006 that claim the benefits of Ser. No. 10/770,248 filed Feb. 2, 2004 (claiming the benefit of provisional application 60/445,104 filed Feb. 6, 2003). Included for reference in its entirety is Ser. No. 12/455,197, filed on May 28, 2009, that claims the benefit of Ser. No.61/130,225, filed May 29, 2008. Included for reference in its entirety is continuation in part Ser. No. 12/585,695 filed Sep. 26, 2009, that claims the benefit of Ser. No. 11/517,147, filed Sep. 7, 2006 (claiming the benefit if the provisional application 60/719,900, filed Sep. 23, 2005). Included for reference in its entirety is continuation in part Ser. No. 12/583,919, filed Aug. 27, 2009, that claims the benefit of Ser. No. 11/637,450, filed on Dec. 12, 2006 (which claims the benefit of Ser. No. 10/741,346, filed on Dec. 22, 2003.)

### FIELD OF THE INVENTION

The invention relates to controlled release compositions that are environmentally safe and naturally derived that can have antimicrobial, antibacterial, antiviral, and/or antifungal activity as well as providing skin or hair benefit. These compositions can be used as preservatives.

The use of environmentally beneficial materials, especially those that are referred to as “green”, is important in choosing preservative ingredients. The use of natural or naturally derived materials is also very much of interest in topically applied or ingested compositions. Using both green and naturally derived materials in a composition as a preservative having exceptional antimicrobial, antibacterial, antifungal, antiviral and/or skin beneficial properties is therefore also important. If all components of the composition are GRAS (Generally Regarded As Safe) and are approved for food use, the resulting composition could also be ingested with little side effects. Because the use of food grade materials is being promoted as a method of selecting ingredients that would be applied to the skin, compositions using GRAS ingredients would meet that requirement.

### BACKGROUND OF THE INVENTION

Many presently used preservatives are not green and natural or naturally derived. Many are not approved for food use. There is a growing movement in the cosmetics industry to not use ingredients that cannot be eaten. Furthermore, generally used preservatives do not provide sustained release properties which allows continuous release of the preservative at effective levels as it gets used up. Finally, preservatives have not previously been used as slow release carriers for other useful components. If the use of a preservative or self-persevering composition included GRAS ingredients that have an antimicrobial component as well as a naturally occurring component that has skin benefits, then this approach would be beneficial in reducing potential irritation from harsh chemicals while improving skin health.

Although N^{α}—(C1-C22) alkanoyl di basic amino acid alkyl (C1 to C22) ester salts have been known since the 1960's, one of the first patents to recommend these amino acids, specifically for food applications was U.S. Pat. No. 3,825,560 (issued Jul. 23, 1979). A number of derivatives are disclosed including N^{α}-cocoyl-L-arginine ethyl ester pyrrolidone carboxylate and N^{α}-lauroyl-L-arginine methyl ester hydrochloride. Since this publication there has been several more patents issued or published disclosing specifically N^{α}-lauroyl-L-arginine ethyl ester hydrochloride salt (LAE). These include U.S. Pat. No. 5,780,658 that discloses a process to prepare LAE, as well as disclosing its use for food applications. U.S. Pat. No. 7,074,447 B2 discloses an antimicrobial composition comprising LAE with potassium sorbate. U.S. Pat. No. 7,087,769 is another process patent suggesting its use for food. Two patent publications, U.S. 2004/0166082 and U.S. 2004/0175350, disclosure di basic amino acid alkyl ester salts useful for cosmetic applications. U.S. 2004/0254232 covers oral care while U.S. 2004/0265443 covers food. EP 1414394B1 discloses new preservative systems in cosmetic preparations. U.S. 2005/0175747 discloses complexes formed between LAE and various anionic hydrocolloids. Finally a patent publication U.S. 2006/0177540 discloses a synergistic combination of LAE with two other ingredients for food applications, and a WIPO patent application WO/2008/014824 discloses that cationic surfactants derived from the condensation of fatty acids and esterified dibasic amino acids, such as from lauric acid and arginine, in particular the ethyl ester of the lauramide of the arginine monohydrochloride (LAE), may be used for the protection against the growth of the microorganisms. The cationic surfactants of this type are also effective against virus infections. Addition of LAE to cultures of Herpes virus type 1 Vaccinia virus and bovine parainfluenzae 3 virus leads to nearly complete reduction of the virus organisms in these cultures, such effects being observed after 5 and 60 minutes.

The use of a N^{α}-lauroyl-L-arginine ethyl ester salt (“LAE salt”) has a broad spectrum of cidal activity, which can be further extended with an effective amount of a glyceryl monoalkanoate ester (acyl monoglyceride) having from 6 to 14 carbon atoms, e.g. monolaurin, to include effectiveness against HIV, HSV-1, SARS, H1N1, and other virus and fungi. Glyceryl monoalkanoates have a long history of safety and a low toxicity profile.

The literature is replete with numerous references concerning glycerol monofatty acid esters having antiviral and antibacterial activity. The most active monoglycerides consist of those esters formed from saturated fatty acids having from 6 to 14 carbon atoms. U.S. Pat. No. 4,997,851 teaches the use of saturated fatty acids and glycerol monofatty acid esters as effective antiviral agents against the HIV and HSV-1 viruses. They were also active against a variety of gram positive and gram-negative bacteria.

U.S. Pat. No. 5,434,182 discloses the spermicidal, antimicrobial and cytocidal activity of glycerol monofatty acid esters. It discloses the combination of fatty acyl glycerides, a chelating acid, and a surfactant which confer excellent antimicrobial activity for preserving processed meats and for disinfecting poultry carcasses. When only one of these three agents was used, the anti-microbial performance was considerably reduced. U.S. Pat. No. 6,414,023 B1 discloses the use of fatty acid monoglycerides in conjunction with 2,4-dichlorobenzyl alcohol.

John J. Kabara in U.S. Pat. No. 6,638,978 B1 lists a preservative formulation for food and cosmetics consisting of monolaurin (“ML”), caprylic and capric acid mixture, and propylene glycol in an aqueous base. U.S. 2005/0084471 A1 teaches the preparation of a preservative for meat, fruits, and vegetables and for the disinfection of inanimate surfaces. The actives include a propylene glycol C7-C14 fatty acid ester as the major component, a surfactant, and an enhancer. Enhancers include phenolic antioxidants and/or a paraben ester. Lastly, U.S. Patent 2006/0030512 A1 describes a long lasting anti-microbial film comprising a glycerol monoester, an amphoteric surfactant, a chelating agent and a solvent like propyl alcohol plus other incipients. All of the above references are incorporated into the body of our present invention.

### DETAILS OF THE INVENTION

The invention relates to controlled release compositions comprising biocidal salts that have antimicrobial, antibacterial, antifungal, antiviral and/or skin beneficial properties and further that can be considered as green and natural or naturally derived

Our invention relates to biocidal salts that have a maximum water solubility that will allow for a slow release mechanism rather than complete solubility, dependent on the specific application. The salt is formed of:

(a) N^{α}—(C1-C22) alkanoyl di basic amino acid alkyl (C1 to C22) ester cationic molecule; and

(b) an anionic functional monomeric or polymeric ion not generally considered as having significant biocidal activity

where the salt partially dissolves when exposed to an aqueous solution
i. releasing sufficient biocidal ion to exceed the MIC or MBC of the target microbes being controlled and
ii. leaving a residual reservoir of undissolved salt to release more biocide as the biocide is used up or otherwise removed from the environment.

The MIC (minimal inhibitory concentration) is defined as the lowest concentration of an antibiotic which will inhibit the (in vitro) growth of an infectious organism. Results are reported in micrograms per mL or ppm. The MBC (minimal bactericidal concentration) is defined as the lowest concentration of an antimicrobial agent needed to kill 99.9% of the initial organism inoculum.

It has been reported in the literature as follows: the MIC for LAE for Staphylococcus aureus ATCC #6538 is 8 ppm (mg/L); the MIC for LAE for Pseudomonas aeruginosa ATCC #9027 is 32 ppm; the MIC for Aspergillus niger ATCC #14604 is 32 ppm; the MIC for Candida albicans ATCC #10231 is 16 ppm. The MBC has been reported in the literature from a study of adaptation of selected microorganisms toward LAE as follows: ˜40 ppm for Staphylococcus aureus ATCC #6538; ˜75 ppm for Pseudomonas aeruginosa ATCC #9027; ˜40 ppm for Candida albicans ATCC #10231.

The biocidal salt is further characterized as being a salt that is formed by a metathesis reaction between a biocidal cationic molecule with a monomeric or polymeric anionic molecule not generally considered as having significant biocidal activity or by an acid-base reaction between a biocidal monomeric or polymeric free base and a monomeric or polymeric acid not generally considered as having significant biocidal activity capable of protonating the free base. The biocidal salts have been found to be extremely effective against a wide variety of microorganisms, e.g., bacteria, fungi, and viruses. Moreover, the salts have important safety, efficacy and toxicity implications since the biocidal components employed in the preparation of the salts by either a metathesis or an acid-base reaction are typically those that have been approved for use by the EPA or the FDA.

The higher molecular weight salts tend to have limited water solubility depending on the application and therefore may have prolonged extended-release properties. The extended release and also possible increased substantivity are due to the charged nature of the species and the slow dissociation of the salts after addition to a substrate, coating, etc. These may be prepared either prior to inclusion into a specific cosmetics or dermatological formula but also as an in situ reaction while making the formula. For many, but most certainly not all, applications, it is desirable to apply the salts in the form of emulsions, nano-emulsions, microemulsions, gels, dispersions or creams.

Specifically the compositions of interest utilize as the cationic portion of the salts a biocide formed from acyl dibasic amino acid esters of L-arginine, L-histine, L-lysine, and L-tryptophan. For the purposes of this invention, we define dibasic amino acids as having two cationic moieties, such as having two primary amine groups (lysine), an amine and guanide group (arginine), a primary amine and an imidazole group (histidine) and a primary amine and an indole group (tryptophan). A preferred cationic species is N^{α}-lauroyl-L-arginine ethyl ester. A preferred embodiment is either a salt or combination of salts of N^{α}—(C1-C22) alkanoyl di basic amino acid alkyl (C1 to C22) ester salts.

The use of N^{α}-lauroyl-L-arginine ethyl ester hydrochloride salt (“LAE”) as the cation in the controlled release biocidal salt has several advantages over other antimicrobials like:
Cost effectiveness
Non-toxic, GRAS approved
Broad spectrum of cidal activity, which can be further extended with monoglyceride fatty acids to include HIV, HSV-1, and other virus and fungi.
Makes unique emulsions, e.g. acts as a surfactant
Inhibits bacteria from attaching to surfaces
Neutralizes endotoxins through the L-arginine metabolization
Thermally stable
pH stability from 3 to 7

Salts made from LAE can have skin and hair conditioning and emollient characteristics, can act as self preserving ingredients while delivering skin or hair benefits, and can also make emulsions. These are truly multifunctional ingredients.

As mentioned above, the biocidal salts employed in this invention are characterized as having a controlled release characteristics. This is due to their limited solubility which results in sufficient biocidal cation being released to kill microbes but leaving excess undissolved salt to act as a reservoir for future release as the biocidal cation is used up. The salts are composed of an N^{α}—(C1-C22) alkanoyl di basic amino acid alkyl (C1 to C22) ester cationic molecule with a monomeric or polymeric anionic molecule of a skin benefit agent. These can be formed for example by an acid base reaction between the cationic base and anionic acid or by metathesis of salts from each.

The anionic molecule may be carboxylic; hydroxy carboxylic; .beta.-keto carboxylic; or phenolic. Suitable carboxylics include adapalene, azelaic, isotretinoin, pantothenic, retinoic; tretinoin and undecylenic. Suitable hydroxy carboxylics include gluconic, glycolic, glyceric, lactic and salicylic. The phenolic may be, e.g., hexylresorcinol or thymol. The anionic molecule may also be a natural or naturally derived molecule, e.g. a citrate, etc., a long chain fatty acid, e.g. omega-3, etc., or similar.

To be of benefit the skin anionic component in the controlled release salt could include vitamin C and its derivatives (salts or esters), e.g. palmitates, phosphates such as magnesium ascorbyl phosphate and sodium ascorbyl phosphate, and tetrasubstituted lipophilic ascorbates, etc., vitamin A and its derivatives, e.g. palmitates, etc., essential fatty acids, e.g. omega acids including 3, 6, and 9 types, including linolenic acid, arachidonic acid, oleic acid, (C18, unsaturated), linoleic acid (C18, polyunsaturated), eicosapentaenoic acid (also known as “EPA”), and docosahexaenoic acid (also known as “DHA”), .etc., alpha hydroxy acids and their derivatives (esters and salts), e.g. alpha hydroxy acetic acid (also known as “glycolic acid”), alpha hydroxypropionic acid (also known as “lactic acid”), alpha hydroxytetranoic acid, alpha hydroxyhexanoic acid, alpha hydroxyoctanoic acid (also known as “alpha hydroxy caprylic acid”), alpha hydroxynonanoic acid, alpha hydroxydecanoic acid, alpha hydroxyundecanoic acid, alpha hydroxydodecanoic acid (also known as “alpha hydroxy lauric acid”), alpha hydroxytetradecanoic acid, alpha hydroxyhexadecanoic acid, alpha hydrox octadecanoic acid, alpha hydroxyoctaeicosanoic acid, etc., beta-hydroxy acids and their derivatives (esters and salts) Hydroxy acids are organic molecules containing at least one carboxylic acid function and at least one hydroxyl group in addition to the carboxyl. Any hydroxy acid which has skin or hair benefits can be used. The hydroxy acid can be an alpha, beta, gamma, delta, epsilon or omega hydroxy acid. If the antimicrobial component of the composition is considered “Green and Naturally Derived”, this is preferable to those that prefer natural materials as preservatives in their cosmetics, food, etc. We include aromatic carboxylic acids with phenol groups, such as salicylic acid, as useful for this invention. While such molecules are not, strictly speaking, considered to be hydroxy acids, however, because the phenol group increases the molecule's acidity, these acids can be useful for some skin benefit applications such as exfoliation.

These salts can also leach out from a substrate that has incorporated the salts, e.g. a food packaging film, a coating, a dental appliance, an adhesive, etc., and deliver antimicrobial activity to the adjacent surfaces and surroundings. Table 1 refers to beef that was packaged in plastic film that is used for food packaging. The LAE-lactate salt at 1% level gave a 1.67 log reduction at 42 days compared to a negative control.

Using a preservative that includes a skin or hair benefit agent that is green and natural can contribute to improving the health of skin or hair of an individual or other mammal that uses or touches a material that contains a preservative of this invention. Foods, cosmetics, or other materials that require preservation and that are packaged using this invention, either by adding the preservative to the food or other material itself, incorporating the preservative into the packaging film or materials, or coating the preservative onto the packaging can contribute to the consumer's wellbeing. Coatings, adhesives, etc. that incorporate this invention can also help contribute to the well being of the individual whose skin or hair comes in contact with the compositions of this invention.

### Enhancement Agents

We have also found that the activity of slow release biocides of this invention can be enhanced with the addition of a monoglyceride fatty acid, e.g. monolaurin, etc., to increase the antimicrobial and antifungal activity for use as a preservative or in combination with other recognized preservatives.

When virucidal activity needs to be enhanced, certain glycerol monofatty acid esters are added. These which have a carbon chain length of C6-C14 are preferred, and C12 (lauryl) is most preferred. The latter is known as monolaurin. The usage of monolaurin for enhancement was found to be from about 1 to about 10 wt. percent, based on the di basic amino acid ester salt derivative. More preferably a range of about 1.5 to about 5.0 wt. % monolaurin is very effective.

Since monolaurin is not very water soluble, a surfactant or cosolvent is required to form a stable solution, emulsion, nanoemulsion, or microemulsion. In general non-ionic, amphoteric, and cationic surfactants with an HLB between 8-20 have been found to be useful. When making a microemulsion a cosolvent is required. Some useful cosolvents are ethanol, methanol, isopropanol, propylene glycol, and other hydroxylic solvents. The surfactant is useful in the range of 1 to 10 wt. percent based on the antimicrobial agents.

The controlled release biocidal salts of this invention may be prepared using either metathesis or acid base reactions. As noted in the McGraw-Hill Dictionary of Scientific and Technical Terms (5.sup.th Edition, 1994), metathesis is a reaction involving the exchange of elements or groups as in the general reaction: AX+BY⇄AY+BX. An alkali or alkaline earth metal (e.g., Na, K, Li, Ca, etc.) salt of the selected anionic monomer or polymer is formed by reacting it with an equivalent amount of an alkali or alkaline earth metal hydroxide in water or water-alcohol solution. An acid salt, e.g., acetate, hydrohalide, gluconate, sulfate, etc. of the selected free base monomer or polymer is formed by reacting it with an equivalent amount of an acid such as acetic, hydrochloric, hydrobromic, gluconic acid, sulfuric, etc. in water or water-alcohol solution.

Thereafter, an equivalent amount of the aqueous alkali or alkaline earth metal salt solution of the selected anionic monomer or polymer is mixed with the aqueous acid salt solution of the selected cationic monomer or polymer.

It is preferred to use an acid-base reaction to prepare the desired complex if the selected monomeric or polymeric acid is capable of protonating the selected monomeric or polymeric free base. The use of the acid-base reaction avoids the necessity of forming an alkali metal salt of the selected anionic monomer or polymer and the acid salt of the selected biocidal cationic monomer or polymer and having to dispose of the salt byproduct. An amine containing free base is preferred. In order for the acid-base reaction to proceed, the acid component must have a transferable proton (Pka) to a basic (Pkb) molecule. Preservative Applications

The following is a representative list of some of the numerous possible preservative applications of the biocidal salts of the invention. It should be understood that this list is presented for illustrative purposes only and does not represent any limitation as to possible applications. It should be further understood that it is within the purview of this invention that the products described below may be combined with conventional antioxidants, antibacterial agents, antifungal agents, hormones, vitamins, antioxidants, hydroxy acids, cleansers, soaps, shampoos, silicones, biocides, humectants, emollients, synthetic or natural oils, deodorizers, perfumes, colorants, preservatives, plant extracts, etc.
(1) skin and hair care products, e.g., sunscreens; suntan lotions; after-sun gels, lotions and creams; antiperspirants; deodorants (solutions, powders, gels, roll-ons, sticks, sprays, pastes, creams, lotions); cleansing creams; skin conditioners; skin moisturizers; protectants; skin aging products; skin wrinkle reduction products; products for treatment of acne; products for treatment of rosacea; age-spot reduction products; stretch-mark reduction products; pimple treatment products, skin soothing products; skin infection and lesion treatment products; skin-redness reduction products; stretch-mark reduction products; varicose and spider-vein reduction products; lotions; oils; hand/body creams; shaving gels/creams; body washes; liquid and solid soaps; blood microcirculation improvement products, cellulite reduction products, body toning products, skin penetration enhancers; skin whitening products; cosmetics; shampoos; shower gels; bubble baths; hair treatment products, e.g., medicated shampoos, mousses, waxes, conditioners, styling agents, lotions, pomades, spray gels, hair dyes and tints, colorant and non-colorant rinses, detangling lotions, hair curling and hair straightening products, hair wave products, etc.; hand (or mechanical) dishwashing compositions; hand sanitizers; and disinfectants; lipsticks and lip balms; salves; collodion; impregnated patches and strips for skin treatment; skin surface implants; impregnated or coated diapers; and the like.
(2) dental care materials: mouthwash; dentifrice; dental floss coated and/or impregnated with the composition; protective coating for teeth; toothbrush bristles coated and/or impregnated with the composition; orthodontic appliance coated and/or impregnated with the composition; orthodontic appliance adhesive; denture appliance coated and/or impregnated with the composition; denture appliance adhesive; endodontic composition coated and/or impregnated with the composition; composite-type dental restorative materials; dental cement; dental liner; dental bonding agent; and the like.
(3) foods and food products: food-stuffs; animal feed-stuffs; grains; breads; bakery products; confectionary; potato products; pasta products; salads; soups; seasonings; condiments; syrups; jams, jellies and marmalades; dairy products; egg-based products; meats and meat-based products; poultry and poultry-based products; fish and fish-based products; crustaceans and crustacean-based products; fresh and dried fruit products; vegetables and vegetable products; greens; salads; sauces; beverages, e.g., wines, tea extracts, beers, juices; and the like.
4) plastics and miscellaneous products, coated and/or impregnated with the composition, including: medical items, e.g., thermometers, catheters, surgical sutures, blood lines, implants, bandages, surgical dressings, surgical apparel, respirators, etc.; food packaging; drug and cosmetic packaging; eating utensils; shower curtains; bath mats; sponges; mops; toilet seats, rubber gloves; contact lenses; hearing aids; shelving paper; carpet pads; pool covers; animal bedding and cat litter; computer covers and computer keys; doorknobs; tampons and sanitary napkins; dental chairs; dryer sheets; dishcloths; paints and coatings; powdered, liquid, gel and spray cleaners for floors sinks, counter-tops, walls, tiles, floors, carpets; deodorizing liquids, solids, sprays, gels and powders; filters; foams; hair brushes; combs; diaper rash preventer; plasma bag treatment; disposable glove treatment; additive to pasteurized cow milk; additive to blood sample tubes to inactivate HIV, HCMV, and other viruses (safety measure for lab technicians and healthcare providers); additives for condoms, band-aids, or bandages; additive for paint; or animal or plant treatment for microbial infections; and the like.
(5) fibers and fabrics coated and/or impregnated with the composition, including natural and synthetic fibers and fabrics manufactured from such fibers; wipes, cloths; surgical gauze; crib covers; bassinet covers; bed linens; towels and wash cloths; tents; draw sheets; cubicle curtains; shower curtains; wall coverings; wood and wood products; hospital clothing such as examination robes, physicians' coats, nurses uniforms, etc.; apparel; paper, non-woven fabric, knitted fabric, woven fabric, brick, stone, plastic, polymer, latex, metal, tile, walls, floors, gurneys, tables, or trays; shoes and the like. Other potential applications disclosed are facial cream (as an acne treatment), bactericidal, fungicidal, virucidal; shampoo, hand lotion; athlete's foot medication (ointment, powder, soap); candies (for sore throat, bad breath, recurrent herpes); ointment or foam spray (for genital herpes legion treatment); shaving cream; mouth wash; after shave lotions; lip balm; paste.

### Formation of Emulsions of the Salts

As mentioned above, the salts employed in the invention have limited water solubility. Therefore, for many preservative applications, it is desirable to utilize the salts in the form of emulsions, nanoemulsions or microemulsions. These emulsions can be prepared using any suitable procedure known to those skilled in the art. However, it should be noted that emulsifying agents used in this invention should be compatible with the limited solubility salts of the invention. Generally nonionic and amphoteric surfactants or combinations of one or more nonionic, one or more amphoteric surfactants can be utilized to provide satisfactory results. Highly charged anionic surfactants may less desirable since they have the potential to reduce the bioactivity of the salts by causing some degree of precipitation of a more insoluble cationic biocide salt, thereby lessening the effectiveness of the salts. It has also been found that cationic phospholipids, preferably in combination with nonionic and/or amphoteric surfactants are effective in the formation of microemulsions or emulsions of the salts.

The following surfactants have been found to be effective in the formation of emulsions, nanoemulsions and microemulsions of the salts: amphoteric amidobetaines; nonionic polyethoxylated sorbitol esters, polycondensates of ethylene oxide-propylene oxides (polyoxamers), polyethoxylated hydrogenated castor oils, and certain cationic phospholipids.

Suitable examples of amidobetaines include cocoamidoethyl betaine, cocoamidopropyl betaine; and mixtures thereof. Other suitable amphoteric surfactants include long chain imidazole derivatives such as the product marketed under the trade name “Miranol C2M” by Rhodia and long chain betaines such as the product marketed under the trade name “Empigen BB” by Huntsman Corporation, and mixtures thereof.

Suitable nonionic surfactants include polyethoxylated sorbitol esters, especially poly-ethoxylated sorbitol monoesters, e.g., PEG sorbitan di-isostearate, and the products marketed under the trade name “Tween” by ICI; polycondensates of ethylene oxide and propylene oxide (polyoxamers), e.g., the products marketed under the trade name “Pluronic” by BASF; condensates of propylene glycol; polyethoxylated hydrogenated castor oil such as the products marketed under the trade name “Cremophors” by BASF; and sorbitan fatty esters marketed by ICI. Other effective nonionic surfactants include the polyalkyl (C.sub.8-C.sub.18) glucosides.

Suitable cationic surfactants include D,L-pyrrolidone-5-carboxylic acid salt of ethyl-cocoyl-L-arginate (CAE) marketed by Ajinomoto, and cocoamidopropyl (PTC), lauramidopropyl PG diammonium chloride phosphates and the like marketed by Uniqema.

The choice of an effective surfactant system will differ somewhat for each biocidal complex. The choice will depend on the surfactant(s)' hydrophilic-lipophilic balance (HLB) to form a stable small particle micelle in an aqueous or aqueous-cosolvent medium.

Other adjuvants useful in formulating the biocidal salts in o/w or w/o type creams, gels, lotions and the like include: polyether-modified silicone, cyclic silicone, methyl polysilicone, polyoxyethylene castor oil, cetostearyl alcohol, neopentyl glycol dicaprate, sorbitan monostearate, polyvinyl alcohol, glycerin, “Carbox”, glyceryl ether, cholesteryl isostearate, ethanol, isopropanol, glycerol monostearate PEG 100 stearate, hydroxymethyl cellulose, cetyl alcohol, lauryl glucoside and the like.

The following examples shall serve to illustrate the various embodiments of the invention. The examples are given solely for the purpose of illustration and are not to be construed as limitations of the present invention since many variations are possible without departing from the spirit and scope of the invention. Unless otherwise indicated, all parts and percentages are on a weight basis.

The preceding specific embodiments are illustrative of the practice of the invention. It is to be understood, however, that other expedients known to those skilled in the art or disclosed herein may be employed without departing from the spirit of the invention or the scope of the appended claims.

Table 1 Antimicrobial Activity of Compounded Films:

Two beef loins from a slaughterhouse were tested in a heat sealed package simulating food packaging. The LLDPE (linear low density polyethylene) film was compounded with 1% w/w LAE-lactate biocidal salt. The results are reported in Table 1. The log reduction values for bacteria that are part of the background flora of raw meat for both beef samples after 42 days indicate that there is a release of the preservative salt from the compounded LLDPE plastic film to the surrounding beef surfaces

TABLE 1
Antimicrobial Activity
of Compounded Films
Containing LAE Derivatives

| | | | | | | | Day 42 | |
|---|---|---|---|---|---|---|---|---|
| | | | | | | | Log | Day 42 |
| | | Log CFU/g | | | | | Reduction | % Reduction |
| Sample ID | Days | 7 | 14 | 21 | 28 | 42 | Vs. control | Vs. Control |
| Ioin film A | | 2.82 | 4.68 | 5.63 | 7.06 | 7.24 | 0 | 0 |
| (control) | | | | | | | | |
| Ioin film B | | 2.31 | 3.68 | 3.97 | 5.32 | 5.57 | 1.67 | 97.9 |

Table 2: Beef Purge Test:

Two beef tenderloins containing a large quantity of purge (defined as liquid surrounding raw meats; meat soaker pads are used to attempt to trap these liquids)) were purchased at the local store. The purge was isolated from the packages and transferred to a series of test tubes, 10 ml of this purge per tube. To the test tubes, was added either 0.5 g of LAE and LAE salts. The samples were mixed on the vortex mixer, and serial dilutions were then plated on petrifilm for time 0 readings. The test tubes were placed in the refrigerator, and plated on petrifilm again at 24 hr and 48 hr. The beef purge test contained natural aerobic bacteria. LAE HCl was tested at 0.5 g/10 ml w/v, and showed immediate positive results, with reduced bacterial growth over the entire 48 hours. The LAE-monolaurate salt also showed significantly reduced bacterial growth versus the control purge sample, but was a bit slower. Both samples showed>99% reduction in bacterial counts after 48 hours. The results are reported in Table 2.

TABLE 2
Beef Purge Test with LAE-HCl and LAE-monolaurate

| | | |
|---|---|---|
| Sample ID | Avg. CFU/g | % Reduction |

0 Hours

| | | |
|---|---|---|
| 5% w/w LAE-HCL | <1.000E+02 | 99.766 |
| 5% w/w LAE-Monolaurate | 3.89E+04 | 8.798 |
| beef purge control | 4.27E+04 | — |

24 Hours

| | | |
|---|---|---|
| 5% w/w LAE-HCL | <1.000E+02 | 99.99 |
| 5% w/w LAE-Monolaurate | 8.91E+04 | 90.88 |
| beef purge control | 9.77E+05 | — |

48 Hours

| | | |
|---|---|---|
| 5% w/w LAE-HCL | <1.000E+02 | 99.999 |
| 5% w/w LAE-Monolaurate | 2.82E+03 | 99.96 |
| beef purge control | 7.08E+06 | — |

The results in Table 2 indicate that the LAE salt (LAE-monolaurate) performed as well as LAE-HCl after 48 hours. The slow release characteristic also is evident by comparing LAE to the LAE salt after 24 hours versus 48 hours. For preservation, this data shows that LAE salts can have equal preservation characteristics.

Table 3: Aloe Vera Food Grade Preservation Test Method

Procedure: Aloe gel prepared as 50% dilution and dispensed 20 g per container/jar; inoculum prepared as 48 hour yeast cultures in tsb and A. niger spore suspension; inoculum pooled and 100 ul added to each jar; preservatives prepared as 1% solutions and added to each prelabeled jar (see description below) 100-400 ul; samples are mixed well and stored at selected temperature; recoveries are performed at selected intervals (1:10 dilutions plated with SDA);

Prelabeled plates are incubated at 25-30 degrees C. for 48-72 hours;

Plates removed from incubation, are counted, cfu recorded and converted to log values;

Log values are subtracted from the control, and plotted on a time graph and reported.

The results in Table 3 show that after two rechallenges, the LAE-lactate salt was superior to the LAE-HCl. The LAE-HCl showed a decrease at d28 versus the LAE-lactate salt, and almost no reduction at d35 after the second rechallenge, compared to a 4.0 log reduction for LAE-lactate salt, while the LAE-lactate salt continued to exhibit a log reduction even at d42. This proves the slow release concept of the LAE salt.

TABLE 3
Aloe vera preservation test
rechallenges at d21, d35

| | | Log reduction | | | | | | | |
|---|---|---|---|---|---|---|---|---|---|
| | | d0 | | | | d21 | | d35 | |
| | pH = 4-4.5 | challenge 7 logs | d2 | d7 | d14 | rechall. | d28 | rechall. | d42 |
| PPM ACTIVE | DAY | d0 | d2 | d7 | d14 | d21 | d28 | d35 | d42 |
| 1725 ppm | LAE-HCl | 0 | 4.7 | 6.0 | 5.8 | 6.0 | 1.7 | 0.0 | 0.0 |
| 1500 ppm | LAE-lactate salt | 0 | 3.7 | 6.0 | 6.0 | 6.0 | 3.9 | 4.0 | 0.7 |
| 1500 ppm | Lactic Acid | 0 | 1.0 | 0.2 | 0.0 | 0.0 | 0.0 | 0.0 | 0.0 |
| 0 | control-no preservative | 0 | 0.6 | 0.2 | 0.0 | 0.0 | 0.0 | 0.0 | 0.0 |

1. A controlled release biocidal composition comprising: a complex characterized as being a salt, wherein the salt is formed by a reaction between
a) a N^{α}—(C1-C22) alkanoyl di basic amino acid alkyl (C1 to C22) ester biocidal cationic molecule and
b) a monomeric or polymeric anionic molecule not generally considered as having significant biocidal activity, and further characterized wherein the complex partially dissolves when exposed to an aqueous solution releasing sufficient biocidal ion to exceed the MIC or MBC of the target bacteria being controlled, and further characterized as leaving a residual reservoir of undissolved complex to release more biocide as the biocide is used up or otherwise removed from the environment.

2. The controlled release biocidal composition of claim 1, where the cationic portion of complex is N^{α}-lauroyl-L-arginine ethyl ester.

3. The controlled release biocidal composition of claim 1, which tends to adhere easily to skin and other surfaces.

4. The controlled release biocidal composition of claim 1, which releases at least 20 ppm biocidal ion when dissolved in water, with the excess salt present remaining undissolved for subsequent release when the biocidal ion is used up or removed from the environment.

5. The controlled release composition of claim 1 wherein the anionic molecule is selected from the group consisting of carboxylic moieity, hydroxy carboxylic moieity, phenolic moieity, enol moieity, acidic amino acid moieity, protein moieity having a residual negative charge, phosphato moieity on nucleotides of DNA, anionic phospholipid moieity, bisphosphonate moieity, phosphonate moieity, sulphate moieity, and sulphonate moieity.

6. The controlled release biocidal salt of claim 5 wherein the phenolic molecule is resorsinol.

7. The controlled release biocidal composition of claim 5 in which the enol moieity is selected from the group consisting of ascorbic acid (vitamin C), its esters, and its phosphated salts.

8. The controlled release composition of claim 5 wherein the carboxylic moieity is selected from the group consisting of adapalene, isotretinoin, pantothenic acid, retinoic acid, tretinoin, aldobionic acids (ABAs), undecylenic acid, alpha, beta, and alpha, beta keto carboxylic acids, and azelaic acid.

9. The controlled release composition of claim 5 wherein the carboxylic moieity is selected from the group consisting of alpha hydroxy acids.

10. The controlled release composition of claim 9 in which the alpha hydroxyl acid moieity is selected from the group consisting of glycolic acid, lactic acid, tartaric acid, mandelic acid, malic acid, citric acid, gluconic acid, glyceric acid, and glyoxylic acid

11. The controlled release biocidal composition of claim 5 in which the carboxylic acid moieity is salicylic acid.

12. A composition comprising
a) a controlled release biocidal composition comprising a complex characterized as being a salt, wherein the salt is formed by a reaction between i) a N^{α}—(C1-C22) alkanoyl di basic amino acid alkyl (C1 to C22) ester biocidal cationic molecule and ii) a monomeric or polymeric anionic molecule not generally considered as having significant biocidal activity, and further characterized wherein the complex partially dissolves when exposed to an aqueous solution controlled, and further characterized as leaving a residual reservoir of undissolved complex to release more biocide as the biocide is used up or otherwise removed from the environment, and
b) an effective amount of one or more saturated C6-C14 fatty acid monoglycerides.

13. The controlled release biocidal composition in claim 1, where said dibasic amino acid is arginine, lysine, histidine or tryptophan.

14. The use of the controlled release biocidal composition as described in claim 1 as a preservative in food and food products, perishable items, packaging, plastics, medical applications, cosmetics, coatings, dental care compositions and products, wound care compositions and products, dermatological care products, surgical soaps, animal care products, woven fabrics, knit fabrics, nonwoven fabrics, foams, apertured films, paper, surgical soaps, surgical gowns, hospital gowns, microbicides, wood and wood products, construction materials, plasterboard, and virucides.

15. A controlled release biocidal composition as described in claim 12, wherein the saturated C6-C14 fatty acid monoglycerides, is glycerol monolaurate.

16. A controlled release composition as described in claim 15, wherein said glycerol monolaurate is present in the amount of 0.001 to about 2% wt. percent by weight of the composition.

17. The controlled release biocidal composition of claim 1 wherein the salt is formed by a metathesis reaction between an acid salt of N^{α}—(C1-C22) alkanoyl di basic amino acid alkyl (C1 to C22) ester cationic molecule and an alkali or alkaline earth metal salt of the anionic monomer or polymer not generally considered as having significant biocidal activity.

18. The controlled release biocidal composition of claim 1 wherein the salt is formed by a) an acid-base reaction between N^{α}—(C1-C22) alkanoyl di basic amino acid alkyl (C1 to C22) ester free base and b) a monomeric or polymeric acid not generally considered as having significant biocidal activity having insignificant biocidal activity and characterized as capable of protonating the free base, and further characterized as the cationic biocide is present in the form of its free acid having a pKa numerical value of about 8 or less and the anion is present in the form of its free base having a pKb numerical value of at least about 6.

19. The controlled release biocidal composition in claim 18 wherein the amine containing base is N^{α}-lauroyl-L-arginine ethyl ester.

20. The controlled release biocidal composition in claim 1 wherein the anionic molecule is selected from the group consisting of saturated or unsaturated long chain fatty acids.

21. The controlled release biocidal composition in claim 20 wherein the unsaturated fatty acid is selected from the group consisting of linolenic, arachidonic, oleic, (C18, unsaturated), and linoleic (C18, polyunsaturated),eicosapentaenoic acid (EPA), and docosahexaenoic acid (DHA).

22. The controlled release biocidal composition in claim 20 wherein the saturated fatty acid is selected from the group consisting of lauric (C12), myristic (C14, also called Tetradecanoic acid), palmitic (C16, also called Hexadecylic acid), or stearic (C18, also called Octadecanoic Acid).

23. The controlled release biocidal composition in claim 1 for use as a deodorizing, antimicrobial, antifungal, anti-mold, anti-yeast, preservative and antiviral agent.

24. The use of the controlled release biocidal composition as described in claim 14, wherein the preservative is impregnated into the packaging.

25. The use of the controlled release biocidal composition as described in claim 24, wherein the packaging material is for food and food products, cosmetics, perishable items, medical applications, coatings, dental care compositions and products, wound care compositions and products, dermatological care products, surgical soaps, animal care products, wood and wood products, construction materials, and plasterboard.

26. The use of the controlled release biocidal composition as described in claim 25, wherein the salt migrates to the surface of the packaging over time, yielding antimicrobial or antiviral activity.
